(19) United States
(12) Patent Application Publication
(10) Pub. No.: US 2025/0213220 A1
(43) Pub. Date: Jul. 3, 2025

(54) METHOD AND SYSTEM FOR PREDICTING BUTTON PUSHING SEQUENCES DURING ULTRASOUND EXAMINATION

(71) Applicant: KONINKLIJKE PHILIPS N.V., EINDHOVEN (NL)

(72) Inventors: Shiyi Cheng, San Francisco, CA (US); Claudia Errico, Medford, MA (US); Gabriel Ryan Mankovich, Boston, MA (US); Sumit Kumar Shukla, Bangalore (IN); Conner David Pitts, Bothell, WA (US)

(21) Appl. No.: 18/850,097

(22) PCT Filed: Mar. 21, 2023
(86) PCT No.: PCT/EP2023/057215

§ 371 Date: Sep. 24, 2024

Related U.S. Application Data
(60) Provisional application No. 63/323,140, filed on Mar. 24, 2022.

(51) Int. Cl. A61B 8/00; G16H 30/20; G16H 40/60

(52) U.S. Cl. CPC A61B 8/467; A61B 8/4254; G16H 30/20; G16H 40/60

(57) ABSTRACT

A method for performing an ultrasound examination includes obtaining sequences of button pushes performed by a user via a control interface (125) during an exam workflow (S212); differentiating components of the exam workflow based on the button pushing sequences (S213); performing sequence pattern mining of the button pushing sequences based on the differentiated components to extract user specific workflow trends, which include most frequently used button pushing sequences (S214); detecting probe motion of a transducer probe (160) during the ultrasound examination (S215); predicting strings of next button pushes using a predictive model based on at least one previous button push in the user specific workflow trends, where predicting the strings of the next button pushes is triggered by the detected probe motion (S216); and outputting macro buttons corresponding to the predicted strings of next button pushes on the control interface, wherein selection of the macro buttons executes the corresponding predicted strings of next button pushes (S217).

### BACKGROUND

In ultrasound imaging, button pushes and keystrokes entered by the user (e.g., sonographer, physician) are logged and stored in service log files as a sequence of events that capture the workflow narrative of ultrasound imaging examinations from beginning to end. The log files therefore provide insightful information regarding the exam workflows. Log files analytics of the log files help designers improve control panel layout of the ultrasound imaging systems. This improves workflow and custom/dynamic keyboard and touch screen design that mimic users' preferences. However, personalized and smart workflows are still being requested by users for faster and more efficient ways of working during ultrasound imaging examinations.

Currently, ultrasound imaging systems are not configurable to the user's settings and preferences, and no intelligence is applied to speed up the imaging exam workflow with regard to suggesting lists or sequences of buttons that should be pushed to complete the scanning protocol. Expert users may have specific ways of working for certain clinical applications and/or specific patient populations, making general workflow solutions viable, but not user specific. For example, expert users may have different image optimization workflows, while novice users may deviate from the better and faster workflows due to inexperience.

### SUMMARY

According to a representative embodiment, a method is provided for performing an ultrasound examination using an ultrasound imaging system, including a transducer probe and a control interface for controlling acquisition of ultrasound images during the ultrasound examination. The method includes obtaining sequences of button pushes performed by a user via the control interface during an exam workflow, each sequence of button pushes having a corresponding sequence length defined by a number of button pushes in the sequence of buttons; differentiating components of the exam workflow based on the sequences of button pushes, wherein the differentiated components depend on a clinical application of the ultrasound examination; performing sequence pattern mining of the sequences of button pushes based on the differentiated components of the exam workflow to extract user specific workflow trends, where the user specific workflow trends comprise a plurality of most frequently used sequences of button pushes; detecting probe motion of the ultrasound transducer probe during the ultrasound examination; predicting strings of next button pushes using a predictive model based on a at least one previous button push in the user specific workflow trends, respectively, wherein predicting the strings of the next button pushes is triggered by the detected probe motion; and outputting at least one macro button corresponding to the predicted strings of next button pushes on the control interface, wherein selection of the at least one macro button by the user executes the corresponding predicted strings of next button pushes.

According to another representative embodiment, a system is provided for performing an ultrasound examination. The system includes an ultrasound imaging system including a transducer probe and a control interface for controlling acquisition of ultrasound images during the ultrasound examination; a display configured to display the ultrasound images; at least one processor coupled to the ultrasound imaging system and the display; and a non-transitory memory for storing instructions that, when executed by the at least one processor, cause the at least one processor to obtain sequences of button pushes performed by a user via the control interface during an exam workflow, each sequence of button pushes having a corresponding sequence length defined by a number of button pushes in the sequence of buttons; differentiate components of the exam workflow based on the sequences of button pushes, where the differentiated components depend on a clinical application of the ultrasound examination; perform sequence pattern mining of the sequences of button pushes based on the differentiated components of the exam workflow to extract user specific workflow trends, where the user specific workflow trends comprise most frequently used sequences of button pushes; predict strings of next button pushes using a predictive model based on at least one previous button push in the user specific workflow trends, respectively; and at least one output macro button on the display corresponding to the predicted strings of next button pushes on the control interface, where selection of the at least one macro button by the user executes the corresponding predicted strings of next button pushes.

According to another representative embodiment, a non-transitory computer readable medium is provided storing instructions for performing an ultrasound examination. When executed by at least one processor, the instructions cause the at least one processor to obtain sequences of button pushes performed by a user during an exam workflow via a control interface, configured to interface with a transducer probe during the ultrasound examination, each sequence of button pushes having a corresponding sequence length defined by a number of button pushes in the sequence of buttons; differentiate components of the exam workflow based on the sequences of button pushes, where the differentiated components depend on a clinical application of the ultrasound examination; perform sequence pattern mining of the sequences of button pushes based on the differentiated components of the exam workflow to extract user specific workflow trends, where the user specific workflow trends comprise most frequently used sequences of button pushes; predict strings of next button pushes using a predictive model based on at least one previous button push in the user specific workflow trends, respectively; and output at least one macro button on a display corresponding to the predicted strings of next button pushes on the control interface, where selection of the at least one macro button by the user executes the corresponding predicted strings of next button pushes.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of an ultrasound imaging system for predicting button pushing sequences during an ultrasound examination, according to a representative embodiment.

FIG. 2 is a flow diagram showing a method of predicting button pushing sequences during an ultrasound examination using an ultrasound imaging system, according to a representative embodiment.

FIG. 3A is a first part of a schematic diagram showing an example of differentiation of components of an exam workflow during an ultrasound examination, according to a representative embodiment.

FIG. 3B is a second part of the schematic diagram showing the example of differentiation of components of an exam workflow during an ultrasound examination, according to a representative embodiment.

FIG. 4 is a plan view of a control interface in an ultrasound imaging system including macro buttons corresponding to strings of predicted next button pushes, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, the various embodiments described herein provide a system and method for improving ultrasound imaging exam workflows by combining log file analysis to ultrasound images and probe tracking to intelligently predict a string of next button pushes. The improved ultrasound imaging exam workflows are dynamically adapted to each user's way of working and preferences, real-time exam workflows, and clinical applications (reasons for the ultrasound examination).

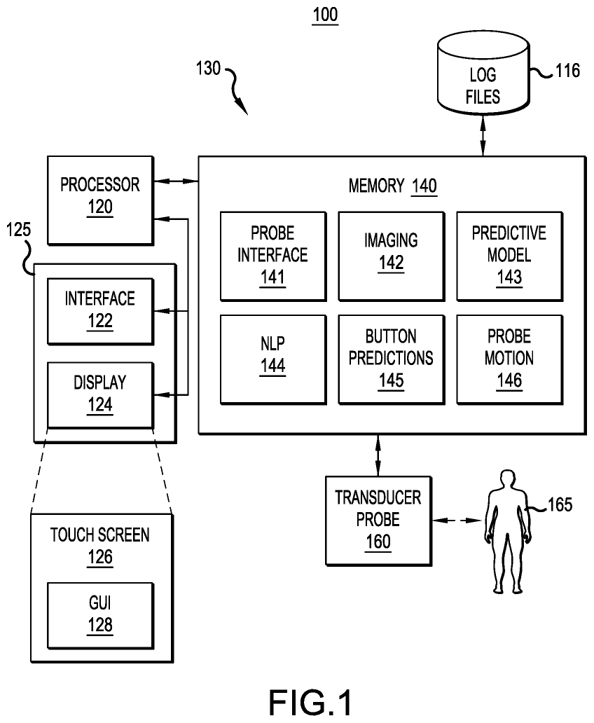

FIG. 1 is a simplified block diagram of an ultrasound imaging system for predicting button pushing sequences during an ultrasound examination, according to a representative embodiment.

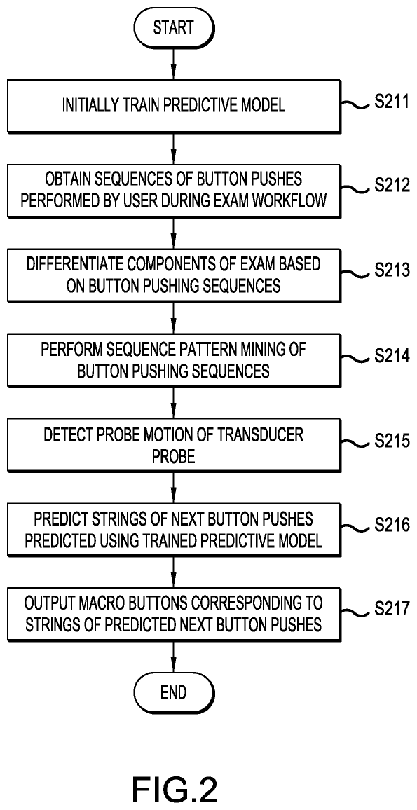

Referring to FIG. 1, the ultrasound imaging system 100 includes a workstation 130 for implementing and/or managing the processes described herein. The workstation 130 includes one or more processors indicated by processor 120, one or more memories indicated by memory 140, user interface 122, and display 124. The user interface 122 and the display 124 may be integrated in a control interface 125 operable by a user to control the ultrasound imaging in accordance with exam workflows, discussed below. The memory 140 stores instructions executable by the processor 120. When executed, the instructions cause the processor 120 to implement one or more processes for predicting button pushing sequences during an ultrasound examination, described below with reference to FIG. 2, for example, as well as to control performance of the ultrasound imaging. For purposes of illustration, the memory 140 is shown to include software modules, each of which includes the instructions corresponding to an associated capability of the ultrasound imaging system 100, as discussed below.

The processor 120 is representative of one or more processing devices, and may be implemented by field programmable gate arrays (FPGAs), application specific integrated circuits (ASICs), a digital signal processor (DSP), a general purpose computer, a central processing unit, a computer processor, a microprocessor, a microcontroller, a state machine, programmable logic device, or combinations thereof, using any combination of hardware, software, firmware, hardwired logic circuits, or combinations thereof. Any processing unit or processor herein may include multiple processors, parallel processors, or both. Multiple processors may be included in, or coupled to, a single device or multiple devices. The term “processor” as used herein encompasses an electronic component able to execute a program or machine executable instruction. A processor may also refer to a collection of processors within a single computer system or distributed among multiple computer systems, such as in a cloud-based or other multisite application. Programs have software instructions performed by one or multiple processors that may be within the same computing device or which may be distributed across multiple computing devices.

The memory 140 may include main memory and/or static memory, where such memories may communicate with each other and the processor 120 via one or more buses. The memory 140 may be implemented by any number, type and combination of random access memory (RAM) and read-only memory (ROM), for example, and may store various types of information, such as software algorithms, artificial intelligence (AI) machine learning models, and computer programs, all of which are executable by the processor 120. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 140 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “nontransitory” specifically disavows fleeting characteristics such as characteristics of a carrier wave or signal or other forms that exist only transitorily in any place at any time. The memory 140 may store software instructions and/or computer readable code that enable performance of various functions. The memory 140 may be secure and/or encrypted, or unsecure and/or unencrypted.

The ultrasound imaging system 100 further includes or interfaces with one or more log files databases for storing information that may be used by the various software modules of the memory 140, indicated by log files database 116. The log files database 116 may be implemented by any number, type and combination of RAM and ROM, for example. The various types of ROM and RAM may include any number, type and combination of computer readable storage media, such as a disk drive, flash memory, EPROM, EEPROM, registers, a hard disk, a removable disk, tape, CD-ROM, DVD, floppy disk, Blu-ray disk, USB drive, or any other form of storage medium known in the art. The log files database 116 is a tangible storage medium for storing data and executable software instructions and are non-transitory during the time data and software instructions are stored therein. The log files database 116 may be secure and/or encrypted, or unsecure and/or unencrypted. For purposes of illustration, log files database 116 is shown as a separate database, although it is understood that it may be combined with, and/or included in the memory 140, without departing from the scope of the present teachings. The log files database 116 may be built as a matter of routine at one or more facilities providing clinical care, storing at least patient demographic and clinical information.

The ultrasound imaging system 100 further includes a transducer probe 160. The transducer probe 160 may include a transducer array comprising a two-dimensional array of transducers, capable of scanning in two or three dimensions, for transmitting ultrasound waves into a subject (patient) 165 and receiving echo information in response. The transducer array may include capacitive micromachined ultrasonic transducers (CMUTs) or piezoelectric transducers formed of materials such as PZT or PVDF, for example. The transducer array is coupled to a microbeamformer in the transducer probe 160, which controls reception of signals by the transducers.

The memory 140 includes a probe interface module 141 for interfacing the transducer probe 160 with the processor 120 to control acquisition of ultrasound images of the subject 165. The probe interface module 141 may include a transmit/receive (T/R) switch coupled to the microbeamformer of the transducer probe 160 by a probe cable. The T/R switch switches between transmission and reception modes, e.g., under control of the processor 120 and/or the user interface 122. The processor 120 also controls the directions in which beams are steered and focused via the probe interface module 141. Beams may be steered straight ahead from (orthogonal to) the transducer array, or at different angles for a wider field of view. The processor 120 may also include a main beamformer that provides final beamforming following digitization. Generally, the transmitting of ultrasound waves and the receiving of echo information is well known, and therefore additional detail in this regard is not included herein.

The processor 120 may include or have access to an AI engine or module, which may be implemented as software that provides artificial intelligence, such as natural language processing (NLP) algorithms, and applies machine learning, such as neural network modeling, described herein. The AI engine may reside in any of various components in addition to or other than the processor 120, such as the memory 140, an external server, and/or the cloud, for example. When the AI engine is implemented in a cloud, such as at a data center, for example, the AI engine may be connected to the processor 120 via the internet using one or more wired and/or wireless connection(s).

The user interface 122 is configured to provide information and data output by the processor 120 and/or the memory 140 to the user and/or to provide information and data input by the user to the processor 120 and/or the memory 140. That is, the user interface 122 enables the user to enter data and to control or manipulate aspects of the processes described herein, and to control or manipulate aspects of the ultrasound imaging. The user interface 122 also enables the processor 120 to indicate the effects of the user's control or manipulation to the user.

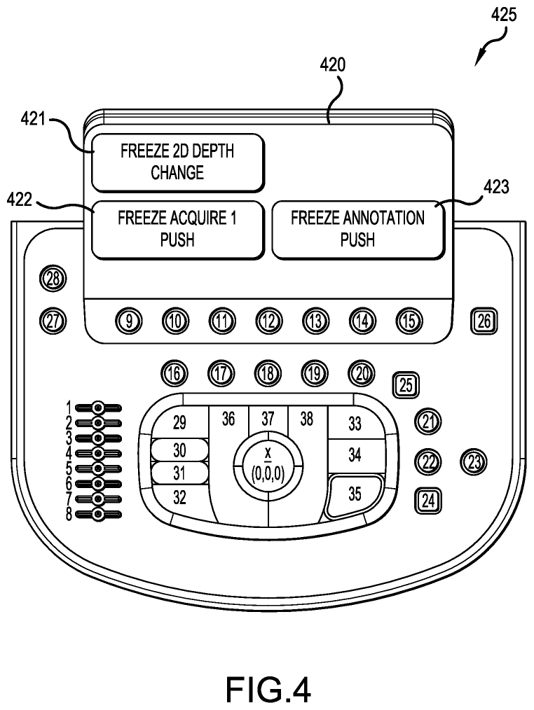

All or a portion of the user interface 122 may be implemented by a graphical user interface (GUI), such as GUI 128 on a touch screen 126 of the display 124, for example, discussed below. The user interface 122 includes push buttons operable (pushed) by the user to initiate various commands for manipulating the displayed image, making measurements and calculations, and the like during the ultrasound examination. The push buttons may be displayed by the GUI 128 on the touch screen 126, or may be physical buttons, examples of which are shown in FIG. 4, discussed below. The button pushes by the user are logged and stored in the log files database 116 as a workflow and utilization script. The user interface 122 may further include any other compatible interface devices for performing ultrasound examinations, such as a mouse, a keyboard, a trackball, a joystick, microphone, a video camera, a touchpad, or voice or gesture recognition captured by a microphone or video camera, for example.

The display 124 may be any compatible monitor for displaying ultrasound images, such as a computer monitor, a liquid crystal display (LCD), an organic light emitting diode (OLED), a flat panel display, or a solid-state display, for example, for viewing internal images of the subject 165. The display 124 includes the touch screen 126 and the GUI 128 to enable the user to interact with the displayed images and features.

Referring to the memory 140, imaging module 142 is configured to receive and process ultrasound images of the subject 165 received from the transducer probe 160 and the probe interface module 141 for display on the display 124. The processing may include band-pass filtering, decimation, I and Q component separation, and harmonic signal separation, for example, as well as signal enhancement, such as speckle reduction, signal compounding, and noise elimination, for example. The current image is displayed on the touch screen 126 as the user manually manipulates the transducer probe 160 on the surface of the subject 165. This enables analysis of the current image by the user for adjusting the position of the transducer probe 160 to acquire desired images, and for making measurements and calculations on the touch screen 126.

Predictive model module 143 is configured to train and apply a predictive model that identifies exam workflows of ultrasound imaging examinations, and predicts one or more next button pushes of button pushing sequences in the exam workflows based on one or more initial button pushes, as describe below. The predictive model takes into account the circumstances of the ultrasound examinations during which the button push sequences were obtained, including different clinical applications.

The predictive model module 143 may be a deep learning neural network, such as a convolutional neural network (CNN), a recurrent neural network (RNN), an artificial neural network (ANN), or a transformer network, for example. The predictive model may be trained using log files analytics of log files in the log files database 116. During training, the predictive model learns sequences of button pushes associated with ultrasound images entered to accomplish various tasks in exam workflows for one or more clinical applications, as discussed below. The training may be performed by obtaining sequences of button pushes performed by users during the exam workflows of previous ultrasound examinations.

NLP module 144 is configured to execute one or more NLP algorithms using word embedding technology to identify button pushes extracted from button pushing sequences entered by the user. The button pushes are typically identified by descriptive text, and must be converted to computer readable data. NLP is well known, and may include syntax and semantic analyses, for example, and deep learning for improving understanding by the NLP module 144 with the accumulation of data, as would be apparent to one skilled in the art. The identified button pushes may be provided to the predictive model module 143 for training and/or to button predictions module 145, discussed below, to convert the natural language identification of button pushes to computer readable data. In various embodiments, all or part of the processes provided by the NLP module 144 and/or the predictive model module 143 may be implemented by an AI engine, for example, executed by the processor 120.

Button predictions module 145 is configured to obtain sequences of button pushes performed by the user via the control interface 125 during an exam workflow, differentiate components of the exam workflow based on the sequences of button pushes, perform sequence pattern mining of the sequences of button pushes based on the differentiated components of the exam workflow to extract user specific workflow trends of most frequently used button pushing sequences, predict strings of next button pushes using the predictive model from the predictive model module 143 based on one or more previous button pushes, and create and output macro buttons corresponding to the predicted strings of next button pushes on the touch screen 126 of the control interface 125. The differentiated components of the exam workflow depend on the clinical application of the particular ultrasound examination. Once the output macro buttons are provided to the touch screen 126, the user may select them, e.g., by touching the designated area of the touch screen 126, to execute the corresponding predicted strings of next button pushes, respectively. The functionality of the button predictions module 145 is described in more detail with reference to FIG. 2, below.

Probe motion module 146 is configured to detect probe motion of the transducer probe 160 during ultrasound examinations. For example, the transducer probe 160 may include one or more sensors, such as electromagnetic (EM) sensors or inertial measurement unit (IMU) sensors. The probe motion module 146 then monitors the probe position of the transducer probe 160 by tracking position data provided by the sensors on the transducer probe 160. The probe motion may be used to trigger the sequence pattern mining and/or the prediction of the next button pushes without further input from the user.

FIG. 2 is a flow diagram showing a method of predicting button pushing sequences during an ultrasound examination by a user using an ultrasound imaging system, according to a representative embodiment. The method may be implemented by the ultrasound imaging system 100, discussed above, under control of the processor 120 executing instructions stored as the various software modules in the memory 140, for example.

Referring to FIG. 2, the method includes initially training a predictive model in block S211 by obtaining sequences of button pushes performed by users during exam workflows of previous ultrasound examinations. As discussed above, the sequences of button pushes may be obtained from previously stored log files in a log files database (e.g., log files database 116) and corresponding images from the exam workflows. Each sequence has a corresponding sequence length defined by the number of button pushes in that button pushing sequence. The previous ultrasound examinations may have been performed by the current user and/or by different users, all of whom are preferably experts in ultrasound imaging techniques.

The predictive model, which may be a deep learning neural network, may be trained using log files analytics. During training, the predictive model learns sequences of button pushes associated with images entered to accomplish various tasks in exam workflows for one or more clinical applications. Training data include button pushing sequences which are extracted and represented by the NLP module 144 from exam workflow log files. The predictive model may take the form of a conditioned generator that uses information of examination circumstances as an auxiliary input signal, and the earlier button pushing sequences (training sequences having later parts masked) as the primary signal, to generate button pushing sequences in the later part of the training data (training sequences having earlier parts masked), through hierarchical computation of the neural network that enables information extraction, processing and combination. The training process is implemented by backpropagating gradients of the neural network weights with respect to a pre-defined objective function that measures the difference between predicted output and ground truth button pushing sequences, which is computed and executed by the processor 120. The learning enables the predictive model to identify relevant exam workflows and to predict one or more next button pushes of button pushing sequences in those exam workflows based on one or more initial button pushes earlier in the same sequences, taking into account the circumstances of the ultrasound examinations during which the button push sequences were obtained, including different clinical applications. At inference time, the predictive model takes examination circumstance information and current button pushing sequences of length one or more performed by the user as input signals to forward propagate through the neural network and compute the predictions of one or more next button pushes.

Notably, although depicted as the first step in the button pushing sequence prediction procedure, initially training the predictive model is not needed in order to perform every ultrasound examination. For example, the training may be performed periodically to update the prediction model, which may then be applied to a number of different ultrasound examinations without further modification. Also, in an embodiment, the results of each ultrasound examination may be used for training the prediction model during subsequent training.

In block S212, sequences of button pushes performed by the user via a control interface (e.g., control interface 125) during a current exam workflow are obtained. Each button pushing sequence corresponds to a component or task of the exam workflow, and has a sequence length defined by the number of button pushes in that button pushing sequence needed to complete the task. The sequence length of the button pushing sequence may be determined by specific gates such as the user freezing or acquiring an image to isolate related sets of events.

The button pushes may be obtained from the control interface and/or from the log files of the ultrasound imaging system. For example, the button pushes may be obtained by accessing the log files database, and pairing the ultrasound images with the log files based on the current exam workflow and a transition state of the ultrasound imaging system, discussed below. The button pushing sequences are thus retrieved and organized so that the context of current exam workflow/images and transition states are attached to the button pushing sequences extracted. The transition states are also relevant for component differentiation discussed below, since they denote which state the system is in for identifying how the button pushing sequences are being used. The button pushing sequences will be used to infer or predict future button pushes using the predictive model previously trained in block S211, as discussed below. The exam workflow is dependent on the ultrasound image content and a clinical application of the ultrasound examination, discussed below.

In block S213, components (or tasks) of the exam workflow are differentiated based on the button pushing sequences and the transition states of the ultrasound imaging system during the exam workflow. Differentiating the components includes converting the button pushing sequences obtained in block S212 to corresponding encoded vectors, clustering the encoded vectors according to usage, and separating the clustered encoded vectors into one of two transition states. The two transition states include “live state” and “frozen state.” In the live state, ultrasound images are displayed in real-time, enabling the user to observe the anatomy of the subject and interactively adjust the positioning of the transducer probe and/or the acoustic settings of the ultrasound imaging system in real-time to attain desired ultrasound images. In the frozen state, the currently displayed ultrasound image is frozen, enabling the user to review the ultrasound image and/or to measure a portion of the anatomy in the ultrasound image, for example. The user may also perform calculations based on the measurements.

With regard to the encoded vectors, the buttons and/or button pushes are effectively “words” for accommodating interaction with the user via the control interface. These words are converted into the encoded vectors, so that they become computer readable sequences. The length of each encoded vector is determined by the number of button pushes in the corresponding sequence of button pushes. In order to convert the button pushing sequences to encoded vectors, the ultrasound images are first paired with log files based on the exam workflow and the transition state of the ultrasound imaging system. The button pushing sequences are then converted to encoded vectors, using NLP-based techniques, for example, discussed above. In an embodiment, user ID of the user may be extracted from the log files as well, so the exam workflow may be customizable.

The component differentiation is affected by the clinical application of the ultrasound examination. Generally, clinical applications include associated scanning protocols based on the anatomy to be examined and the purpose of the examination, where the components of the exam workflow correspond to steps in the scanning protocols, respectively, although the specific exam workflow/button presses may vary to get to complete the protocol. For example, the exam workflow for examining a liver follows a different scanning protocol than the exam workflow for examining a bladder. Also, with regard to imaging the liver in particular, the scanning protocol for a routine study is different from the scanning protocol for fibrosis staging quantification, for example.

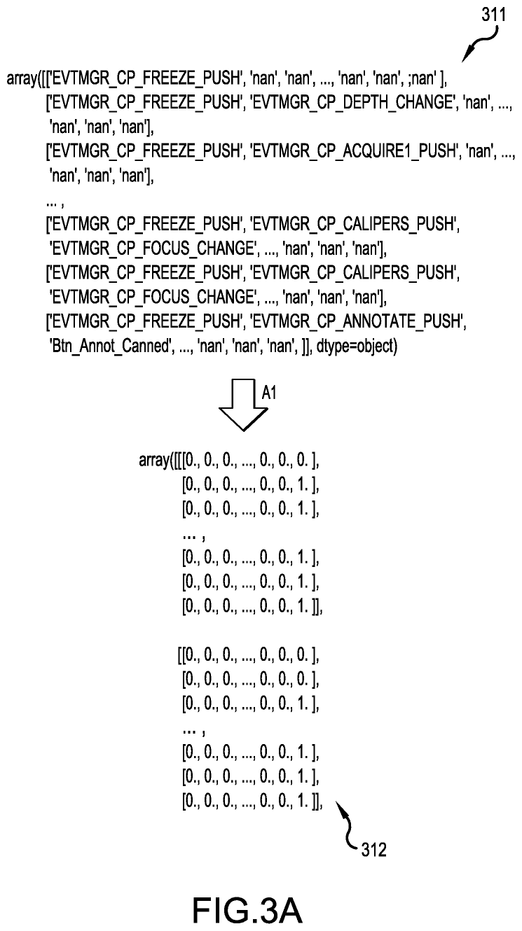

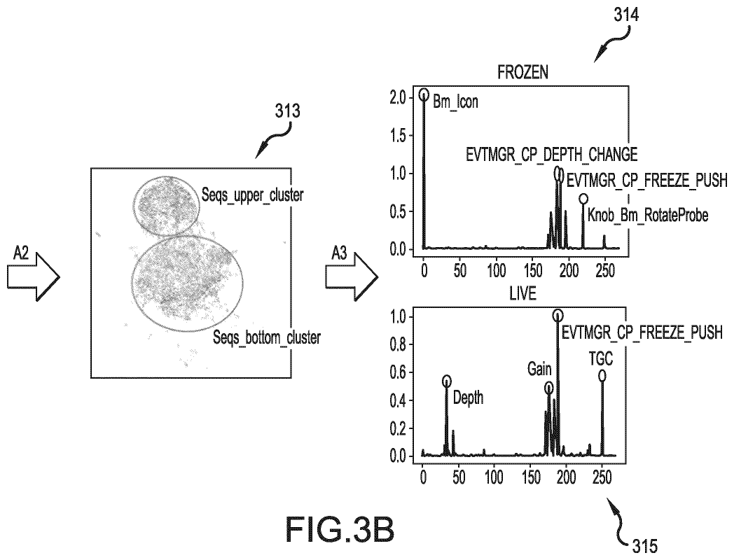

FIGS. 3A and 3B provide a schematic diagram showing an example of differentiating components of an exam workflow during an ultrasound examination, according to a representative embodiment.

FIG. 3A shows illustrative button pushing sequences 311, which are sets of words indicative of respective strings of commands entered by the user for interacting with the ultrasound imaging system. Each set of words, enclosed by a pair of brackets, corresponds to a component of the exam workflow. The button pushing sequences 311 are converted to corresponding encoded vectors 312, indicated by arrow A1. As shown, the encoded vectors 312 are single row (or column) matrices of numbers representing the sets of words in the button pushing sequences 311, respectively. The encoded vectors 312 are computer readable, which enables the subsequent steps in differentiating the components.

The encoded vectors 312 are clustered into clusters 313, indicated by arrow A2 in FIG. 3B. The clustering process is performed using an unsupervised dimension reduction algorithm that projects high-dimensional data onto a two-dimensional manifold while maximally preserving the topological structure of the original set of data points. The demonstrated relative topological structure, which may include two or more clusters, are indicative of different usages or components, dependent on the specific clinical application. In a simplified general case for illustration, two clusters 313, which are denoted as Seqs_upper_cluster and Seqs_bottom_cluster in FIG. 3B, correspond to live and frozen states 314 and 315, respectively. The horizontal and vertical axes of the clusters 313 represent x and y coordinates of the latent vectors in the two-dimensional manifold, so the respective values are relative. In other various cases where the button pushing sequence data are collected across different examination circumstances or applications, the encoded vectors may be clustered according to more than two different usages or components.

The clusters 313 are separated into the frozen state 314 and the live state 315, indicated by arrow A3 in FIG. 3B. This is done by collecting the corresponding original sequence data according to the clusters 313 and counting the frequency of unique button strings per sequence from each cluster, whereafter the clusters 313 can be separated into different states by visualizing and analyzing the frequency plot. In each of the frozen state 314 and the live state 315, the respective button pushes are indicated by frequency, and for purposes of illustration (in each of the two differentiated components), the most frequently used button pushes have been labeled. So, for example, the most frequently used button pushes in the frozen state 314 are Bm_Icon, EVTMGR_CP_DEPTH_CHANGE, EVTMGR_CP_FREEZE_PUSH, and Knob_Bm_RotateProbe, and the most frequently used button pushes in the live state 315 are EVTMGR_CP_FREEZE_PUSH, TGC, Depth, and Gain. For each state, the horizontal axis represents the index of each unique button name, and the vertical axis represents the sequence-wise frequency, which is the average number of incidents of the button name per sequence.

Returning to FIG. 2, in block S214, sequence pattern mining of the button pushing sequences is performed based on the differentiated components of the exam workflow. The sequence pattern mining extracts patterns of the most frequently used contiguous strings of buttons pushed during the ultrasound examination in order to identify user specific workflow trends. Accordingly, the sequence pattern mining may identify K workflow trends from the K most frequent usage patterns, where K is a positive integer. The workflow trends followed by the user may be identified based on the real-time imaging context of the button pushes and the clinical application. Sequence pattern mining is performed on the most current dataset of the user-specific button pushing sequences from past exams, which may be updated after a certain number of new examinations are completed.

As an example, for a clinical application involving an abdominal ultrasound examination of aorta-bifurcation, the sequence pattern mining of the button pushing sequences may result in the following ten workflow trends, ranked from the most frequently used to less frequently used:
1: [‘EVTMGR_CP_FREEZE_PUSH’, ‘EVTMGR_CP_ANNOTATE_PUSH’, ‘SublLabel_Label’, ‘EVTMGR_C_FREEZE_PUSH’]
2: [‘EVTMGR_CP_FREEZE_PUSH’, ‘Bm_Icon’, ‘Bm_Icon’, ‘EVTMGR_CFREEZE_PUSH’]
3: [‘EVTMGR_CP_FREEZE_PUSH’, ‘EVTMGR_CP-ACUIRE1_PUSH’, ‘EVTMGR_CP-ACUIRE1_PUSH’, ‘EVTMGR_CP_FREEZE_PUSH’]
4: [‘EVTMGR_CP_FREEZE_PUSH’, ‘SublLabel_Label’, ‘SublLabel_Label’, ‘EVTMGR_CP_FREEZE_PUSH’]
5: [‘EVTMGR_CP_FREEZE_PUSH’, ‘EVTMGR_CP_DEPTH_CHANGE’, ‘EVTMGR_CP_DEPTH_CHANGE’, ‘EVTMGR_CP_FREEZE_PUSH’]
6: [‘EVTMGR_CP_FREEZE_PUSH’, ‘SublLabel_Label’, ‘Btn_Annot_Canned’, ‘EVTMGR_CP_FREEZE_PUSH’]
7: [‘EVTMGR_CP_FREEZE_PUSH’, ‘SublLabel_Label’, ‘EVTMGR_CP ACUIRE1_PUSH’, ‘EVTMGR_CP_FREEZE_PUSH’]
8: [‘EVTMGR_CP_FREEZE_PUSH’, ‘Btn_Annot_Canned’, ‘SublLabel_Label’, ‘EVTMGR_CP_FREEZE_PUSH’]
9: [‘EVTMGR_CP_FREEZE_PUSH’, ‘Bm_Icon’, ‘Bm_Icon’, ‘Bm_Icon’]
10: [‘EVTMGR_CP_FREEZE_PUSH’, ‘Bm_Icon’, ‘EVTMGR_CP_DEPTH_CHANGE’, ‘EVTMGR_CP_FREEZE_PUSH’]

In an embodiment, the log files database may need to be projected into a smaller space of sub-sequences in order to apply pattern constraints to the sequence pattern mining, such as a gap constraint and a sequence length constraint, for example. The gap constraint ensures that each of the most frequent workflow trends will include a gap between buttons that cannot be contiguously selected by the user in a real ultrasound examination scenario. For example, the Freeze Push button is frequency used by a user to transition from the live state to the frozen state of the ultrasound imaging system, and vice versa. However, even when the Freeze_Push button is frequently used during an examination, it is rare that the user will push it multiple times contiguously. Adding the gap constraint eliminates duplicate or outlier sequences that may contain contiguously multiple same button strings, which makes less sense from the clinical perspectives, thereby improving sequence pattern mining accuracy. The sequence length constraint applies a pre-selected minimum sequence length to the sequence pattern mining based on user input and clinical application context. Generally, smaller button pushing sequences produce more usage patterns, but may be less meaningful in the workflow. So, sequences of fewer button pushes than the pre-selected minimum are not considered when determining the most frequent work flow trends.

In another embodiment, the sequence pattern mining may be performed using weighted button pushes. The weighting may be based on user preferences, which depend on the clinical context, to reflect different exam workflow approaches within a selected workflow trend. For example, the user may prefer a transverse view of certain organs, and a longitudinal view of other organs. The extracted patterns of button pushes are weighted based on the clinical application, as well as clinical context, e.g., where the clinical application may refer to the general exam type being performed and clinical context may refer to the more specific imaging environment the system can recognize. For example, different weights may be assigned to different button pushes when the user is performing an abdominal exam for screening purposes as compared to a follow-up abdominal exam. The different weights are applied subject to the sequence pattern mining to identify favorable trends.

In block S215, probe motion of the transducer probe is detected during the ultrasound examination. When performing the ultrasound examination according to a scanning protocol, the user moves the ultrasound transducer probe on the subject's body surface while viewing the image content to survey the anatomy and to find the best imaging view. When probe tracking is enabled, the movement of the transducer probe is detected, for example, using sensor(s) attached to the transducer probe, as discussed above.

Certain pre-determined movements or patterns of movements may be used to trigger prediction of a sequence of next button pushes for the ultrasound examination. For example, as the user gets closer to the view of interest in the subject, the probe motion becomes more stable (e.g., slower, less frequent movements over smaller distances). Also, the probe motion typically stops at an anatomical landmark(s) indicating the anatomical structure of interest being investigated, where qualitative and quantitative analysis are to be performed. Therefore, the prediction may be triggered based on the detected more stable and/or stopped probe motion.

In block S216, strings of next button pushes are predicted using the predictive model trained in block S211 based on at least one previous button push in the user specific workflow trends, respectively. Generally, the prediction model predicts the next M buttons to be pushed in sequence for the ultrasound examination based on N previous button pushes, where M and N are positive integers.

The predictive model may be an auto-regressive model, for example, and may be used with a decoder. The decoder is configured to decode a sequence of predicted vectors (in the form of matrices), which are computer readable, into a sequence of button strings or names, which are human interpretable. The log files and button pushes associated with the workflow trends provided by the sequence pattern mining are divided into smaller subsets (batches) of button pushing sequences, so that the individual button pushes of each button pushing sequence may be considered. Dividing the log files and button pushes into the subsets may either be “learned” by the predictive model, e.g., during the training process, or “customized” by the user. That is, the predictive model or the user may select (or pre-select) the length of the string for the prediction task (the number of next button pushes to predict). In an embodiment, the user pre-selection may be provided based on user preferences identified using the user ID, discussed above, and realtime image content extraction. Or, the user pre-selection may be retrieved from default settings of the ultrasound imaging system, or entered directly by the user from the control interface, e.g., using a dedicated knob or other input.

For example, when the user makes a pre-selection of N=3 previous button pushes, the predictive model predicts next button pushes based on the most recent three previous button pushes by the user. The accuracy and the confidence with which each next sequence of button pushes is predicted depends on the value of N previous button pushes considered as input and the value of M predicted next button pushes for the length of the predicted sequence. Generally, the larger the number N of previous button pushes, and the smaller the number M of next predicted button pushes, the prediction accuracy and confidence levels increase and vice versa. The predictions may be triggered by certain probe motions, such as the transducer probe making more stable movements or coming to a stop, as discussed above.

In block S217, macro buttons corresponding to the strings of predicted next button pushes are output on the control interface. For example, the macro buttons may be created and displayed on a touch screen of the control interface, along with a label indicating the associated function of the predicted next button pushes. The user is then able to select the macro buttons to execute the corresponding strings of predicted next button pushes. Using the macro buttons leads to faster and more consistent execution of the clinical application specific workflow. In an embodiment, the macro buttons may appear in different colors, which represent different confidence levels associated with strings of predicted next buttons, for example.

FIG. 4 is a plan view of a control interface in an ultrasound imaging system including macro buttons corresponding to strings of predicted next button pushes, according to a representative embodiment. Referring to FIG. 4, control interface 425, e.g., which may correspond to the control interface 125, includes a touch screen 420, which displays the macro buttons corresponding to the strings of predicted next button pushes. The control interface 425 also includes standard slider controls 1-8 and push buttons 9-35 corresponding to predetermined functionality. In the depicted example, the touch screen 420 provides a first macro button 421 labeled Freeze 2D Depth change, a second macro button 422 labeled FreezAcquire1 Push, and a third macro button 423 labeled Freeze Annotation push. Also, in the depicted example, the first and second macro buttons 421 and 422 are displayed in a first color (e.g., green) indicated by light shading to show a high confidence level in the associated strings of predicted next buttons, while the third macro button 423 is displayed in a second color (e.g., yellow) indicated by darker shading to show a medium confidence level in the associated strings of predicted next button.

In accordance with various embodiments of the present disclosure, the methods described herein may be implemented using a hardware computer system that executes software programs stored on non-transitory storage mediums. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although predicting button pushing sequences have been described with reference to exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Also, although predicting button pushing sequences has been described with reference to particular means, materials and embodiments, there is no intention to be limited to the particulars disclosed; rather the embodiments extend to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of performing an ultrasound examination using an ultrasound imaging system comprising a transducer probe and a control interface for controlling acquisition of ultrasound images during the ultrasound examination, the method comprising:
obtaining sequences of button pushes performed by a user via the control interface during an exam workflow, each sequence of button pushes having a corresponding sequence length defined by a number of button pushes in the sequence of buttons;
differentiating components of the exam workflow based on the sequences of button pushes, wherein the differentiated components depend on a clinical application of the ultrasound examination;
performing sequence pattern mining of the sequences of button pushes based on the differentiated components of the exam workflow to extract user specific workflow trends, wherein the user specific workflow trends comprise a plurality of most frequently used sequences of button pushes;
detecting probe motion of the transducer probe during the ultrasound examination;
predicting strings of next button pushes using a predictive model based on at least one previous button push in the user specific workflow trends, respectively, wherein predicting the strings of the next button pushes is triggered by the detected probe motion; and
outputting at least one macro button corresponding to the predicted strings of next button pushes on the control interface, wherein selection of the at least one macro button by the user executes the corresponding predicted strings of next button pushes.

2. The method of claim 1, wherein obtaining the sequences of button pushes comprises:
accessing log files of the ultrasound imaging system; and
pairing ultrasound images with the log files based on the exam workflow and transition states of the ultrasound imaging system.

3. The method of claim 2, wherein the transition states of the ultrasound imaging system comprise a live state for providing ultrasound images in real-time and a frozen state for freezing an ultrasound image enabling the user to review and/or measure a portion of the ultrasound image.

4. The method of claim 2, wherein differentiating the components of the exam workflow comprises:
converting each sequence of button pushes to an encoded vector;
clustering the encoded vectors; and
separating the clustered encoded vectors into the transition states.

5. The method of claim 1, wherein performing the sequence pattern mining comprises:
applying a gap constraint to the extracted user specific workflow trends to skip buttons that are less meaningful in the sequences of button pushes.

6. The method of claim 1, wherein performing the sequence pattern mining comprises:
applying a sequence length constraint to the extracted user specific workflow trends to exclude each of the most frequently used sequences of button pushes that are less than a predetermined minimum sequence length.

7. The method of claim 1, wherein performing the sequence pattern mining comprises:
assigning different weights to the user specific workflow trends based at least in part on the clinical application of the ultrasound examination.

8. The method of claim 1, wherein detecting the probe motion of the transducer probe comprises monitoring the transducer probe using an external camera, and determining the probe motion from images provided by the external camera.

9. The method of claim 1, wherein detecting the probe motion of the transducer probe comprises monitoring the transducer probe using at least one sensor on the transducer probe, and determining the probe motion from position data provided by the at least one sensor.

10. The method of claim 9, wherein the at least one sensor comprises at least one of an electromagnetic (EM) sensor or an inertial measurement unit (IMU) sensor.

11. The method of claim 1, wherein the control interface comprises a touch screen display.

12. The method of claim 11, wherein the at least one macro button is displayed on the touch screen in a color of a plurality of different colors corresponding to a plurality of confidence levels associated with the at least one macro button.

13. A system for performing an ultrasound examination, comprising:
an ultrasound imaging system comprising a transducer probe and a control interface for controlling acquisition of ultrasound images during the ultrasound examination;
a display configured to display the ultrasound images;
at least one processor coupled to the ultrasound imaging system and the display; and
a non-transitory memory for storing instructions that, when executed by the at least one processor, cause the at least one processor to:
obtain sequences of button pushes performed by a user via the control interface during an exam workflow, each sequence of button pushes having a corresponding sequence length defined by a number of button pushes in the sequence of buttons;
differentiate components of the exam workflow based on the sequences of button pushes, wherein the differentiated components depend on a clinical application of the ultrasound examination;
perform sequence pattern mining of the sequences of button pushes based on the differentiated components of the exam workflow to extract user specific workflow trends, wherein the user specific workflow trends comprise a plurality of most frequently used sequences of button pushes;
predict strings of next button pushes using a predictive model based on at least one previous button push in the user specific workflow trends, respectively; and
output at least one macro button on the display corresponding to the predicted strings of next button pushes on the control interface, wherein selection of the at least one macro button by the user executes the corresponding predicted strings of next button pushes.

14. The system of claim 13, wherein the instructions further cause the at least one processor to:
detect probe motion of the transducer probe during the ultrasound examination; and
trigger the predicting of the strings of the next button pushes in response to the detected probe motion.

15. The system of claim 14, wherein the instructions cause the at least one processor to detect the probe motion of the transducer probe by monitoring the transducer probe using at least one sensor on the transducer probe, and determining the probe motion from position data provided by the at least one sensor.

16. The system of claim 13, wherein the instructions cause the at least one processor to obtain the sequences of button pushes by:
accessing log files of the ultrasound imaging system; and
pairing ultrasound images with the log files based on the exam workflow and transition states of the ultrasound imaging system.

17. The system of claim 16, wherein the transition states of the ultrasound imaging system comprise a live state for providing ultrasound images in real-time and a frozen state for freezing an ultrasound image enabling the user to review and/or measure a portion of the ultrasound image.

18. The system of claim 16, wherein the instructions cause the at least one processor to differentiate the components of the exam workflow by:
converting each sequence of button pushes to an encoded vector;
clustering the encoded vectors; and
separating the clustered encoded vectors into the transition states.

19. A non-transitory computer readable medium storing instructions for performing an ultrasound examination that, when executed by at least one processor, cause the at least one processor to:
obtain sequences of button pushes performed by a user during an exam workflow via a control interface, configured to interface with a transducer probe during the ultrasound examination, each sequence of button pushes having a corresponding sequence length defined by a number of button pushes in the sequence of buttons;
differentiate components of the exam workflow based on the sequences of button pushes, wherein the differentiated components depend on a clinical application of the ultrasound examination;
perform sequence pattern mining of the sequences of button pushes based on the differentiated components of the exam workflow to extract user specific workflow trends, wherein the user specific workflow trends comprise a plurality of most frequently used sequences of button pushes;
predict strings of next button pushes using a predictive model based on at least one previous button push in the user specific workflow trends, respectively; and
output at least one macro button on a display corresponding to the predicted strings of next button pushes on the control interface, wherein selection of the at least one macro button by the user executes the corresponding predicted strings of next button pushes.

20. The non-transitory computer readable medium of claim 19, wherein the instructions further cause the at least one processor to:
detect probe motion of the transducer probe during the ultrasound examination; and
trigger the predicting of the strings of the next button pushes in response to the detected probe motion.
